(19) United States
(12) Patent Application Publication
(10) Pub. No.: US 2011/0245899 A1
(43) Pub. Date: Oct. 6, 2011

(54) Solar powered tanning booth

(76) Inventors: John Michael Astin, Corpus Christi, TX (US); Jerry Hubert Willis, Corpus Christi, TX (US)

(21) Appl. No.: 13/066,116

(22) Filed: Apr. 5, 2011

Related U.S. Application Data
(60) Provisional application No. 61/341,697, filed on Apr. 5, 2010.

(51) Int. Cl. A61N 5/06; A01G 9/14

(52) U.S. Cl. 607/91; 47/17

(57) ABSTRACT

A solar powered tanning booth or bed includes a wheeled frame and a readily detachable transparent cover. In cold weather, a person can comfortably enjoy solar tanning inside the booth. By detaching the cover, it can be used in cold or inclement weather to protect fragile plants. With the cover detached, a person can comfortably enjoy solar tanning in warm weather by reclining on the booth. The frame includes a peripheral member having a flat upper surface supporting a normally planar body support panel. A brace under the panel is normally spaced from the panel but loading the panel causes it to sag into load supporting engagement with the brace and become upwardly concave which is a more comfortable situation.

This application is partly based on Provisional Application Ser. No. 61/341,697, filed Apr. 5, 2010, priority of which is claimed and the disclosure of which is incorporated herein by reference.

This invention relates to a tanning booth and more particularly to a solar powered tanning booth which has multiple modes of use.

### BACKGROUND OF THE INVENTION

Most known tanning booths include UV lamps which provide the necessary light to produce tanning of humans. Solar powered tanning booths are known in the prior art as shown in U.S. Pat. Nos. 2,653,612; 4,525,884; 4,719,935; 4,739,763; 4,989,600; 5,088,514; 5,387,230; 5,446,580 and 5,733,314. The advantage of solar powered tanning booths is they do not require UV lamps and a convenient power source. Another feature of solar powered tanning booths is they are designed to be used outside in winter during quite cold climates. The sun's rays, passing through a transparent cover, produce quite pleasant temperatures inside the booth, even when it is very cold outside.

Other disclosures of interest relative to this invention are found in U.S. Pat. Nos. 2,834,351; 3,170,172; 3,741,218; 4,309,616; 4,660,561; 4,683,886; 4,686,719; 4,881,548; 4,995,067; 5,546,678 5,733,314; 6,478,810 and 7,001,414 along with U.S. Printed Patent Application 2010/0154118 and foreign patents WO 90/12940, GB 2,116,685 and Germany 3,715,725.

### SUMMARY OF THE INVENTION

The solar powered tanning booths of the prior art have a number of disadvantages overcome by the disclosed device. All of the prior art devices appear to be bulky and awkward to move. This is a particular disadvantage in situations where the tanning booth includes a transparent cover and is alternatively used in winter as a small greenhouse to protect delicate plants or used as a nursery to start young plants grown from seeds or cuttings. The disclosed device is mounted on a wheeled frame, analogous to a chaise lounge frame, which allows simple and expeditious moving.

Another difficulty with prior art solar powered tanning booths is the transparent cover or lid is not well supported and prone to distort or fail. In the disclosed device, an underlying frame has a flat metal surface supporting a flat metal rim on the lid. Because the frame surface has no upstanding lip, there is no accumulation of rain water as will occur with an upstanding lip or angle. When the lid closes, the metal lid rim is flush with the frame surface thereby stabilizing and supporting the periphery of the transparent lid.

Another advantage of this invention is the flat body support on which the user lies is somewhat resilient. An underlying support is out of engagement with the flat body support so, when loaded, the underlying support becomes upwardly concave and considerably more comfortable than a rigid planar surface.

Another advantage of this invention is the transparent lid is detachable from the underlying frame so the lid and frame can be used separately.

It is accordingly an object of this invention to provide an improved tanning booth and, more particular, an improved solar powered tanning booth having multiple modes of use.

Another object of this invention is to provide a solar powered tanning booth incorporating a improved technique for supporting and stabilizing a transparent cover.

A further object of this invention is to provide a solar powered tanning booth capable of wheeled movement.

These and other objects and advantages of this invention will become more fully apparent as this description proceeds, reference being made to the accompanying drawings.

### BRIEF DESCRIPTION OF THE DRAWINGS

FIG. 1 is an isometric view of a tanning booth, illustrating it a cover closed, as if a person were inside being tanned;

FIG. 2 is an isometric view of the tanning booth of FIG. 1, illustrating the cover open;

FIG. 3 is a cross-sectional view of the tanning booth of FIG. 1, taken substantially along line 3-3 thereof as viewed in the direction indicated by the arrows; and

FIG. 4 is a cross-sectional view of the tanning booth of FIG. 1, taken substantially along line 4-4 thereof as viewed in the direction indicated by the arrows.

### DETAILED DESCRIPTION OF THE INVENTION

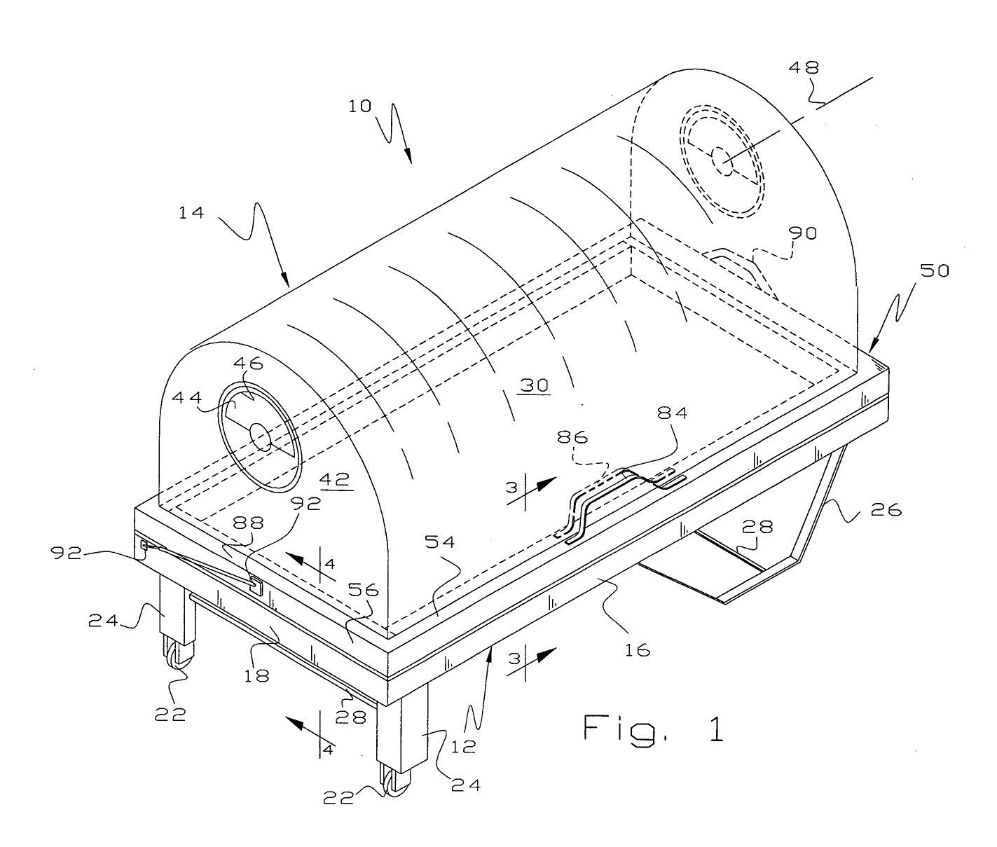

Referring to FIGS. 1-4, a tanning booth or bed 10 comprises, as major components, an underlying frame 12 and a transparent cover or lid 14. The tanning booth 10 can preferably be of the solar powered type in which sunlight passing through the transparent lid 14 provides tanning energy to a person under the lid 14. In this mode of use, the tanning booth 14 may be used in winter or cold weather because sunlight passing through the lid 14 produces a warm environment inside the booth 10, despite it being cold outside.

As will be more fully apparent hereinafter, the lid 14 may be removed from the frame 12 so the frame 12 and lid 14 may be used separately. With the lid 14 detached, the frame 12 may be used during summer where a user lies on the support during daylight to receive tanning rays from the sun because if the user were inside the lid 14, is would be too warm for comfort. With the lid 14 detached, the lid 14 may be used in winter or cold weather as a small portable greenhouse by placing it over temperature sensitive plants to protect them from cold weather.

The frame 12 can be of any suitable material and may be of aluminum which may be anodized, painted or unpainted. The frame 12 includes a pair of elongate structural members 16 extending parallel to a long dimension of the booth 10 and one or more structural members 18 extending perpendicular and welded to the structural members 16 so the frame 12 can be of rectangular shape. The members 16, 18 may be of any suitable shape such as channel, angle, box or the like and may be similar or different in cross-section. The members 16, 18 can provide a flat upper coplanar surface 20 that is free of any upwardly projecting lips or angles which prevents any rainwater from accumulating on the frame 12.

The frame 12 can also include at least two wheels 22 at one end of the frame 12 mounted on standards 24 and wheels or a pair of stands 26 at an opposite end of the frame 12. Suitable spreaders 28 may be provided to strengthen the standards 22 and/or the stands 26. At least one and preferably two braces 30 can extend across the short dimension of the frame 12 and prevent the frame members 16 from spreading apart when a person is supported in the tanning booth 10. The braces 30 can provide another advantage of the tanning booth 10 as explained more fully hereinafter.

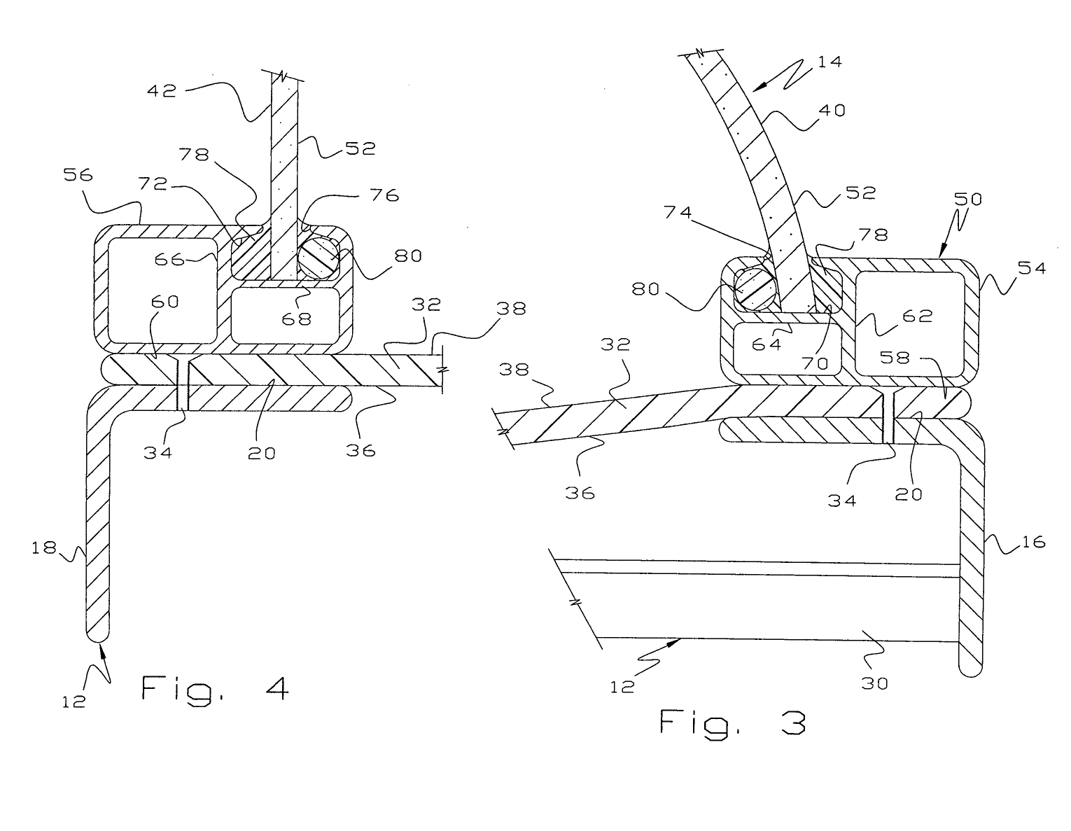

A normally planar panel 32 can be provided to support the weight of a person on the frame 12. The panel 32 may be of any suitable material, including plywood or the like, but may preferably be a polymer sheet known as STARBOARD made by King Plastics Corporation of North Port, Fla. which has the advantage that it does not fade, chalk, discolor, rot or chalk. The panel 32 is of a material and is of a thickness that is somewhat flexible, similar in flexibility to ½″ plywood for purposes more fully explained hereinafter. The panel 32 overlaps the upper surface of the frame members 16, 18 and is connected in any suitable manner, as by the use of threaded fasteners 34 at suitable intervals around the periphery of the frame 12. Because the panel 32 can be normally planar, in an unloaded condition, the bottom 36 of the panel 32 is spaced from and out of load supporting relation to the brace 30. As shown best in dashed lines in FIG. 3, the normal position of the panel 32 is generally parallel to the brace 30. When a person of normal adult weight, i.e. in excess of 100 pounds, lies on the panel 32, the top 38 of the panel 32 bows or assumes a slightly upwardly concave shape shown in FIG. 3 which is considerably more comfortable than a rigid flat bed. As suggested in FIG. 3, the amount of concavity is subject to wide variation and depends on the spacing between the brace 30 and the bottom 36 of the panel 32. This spacing may be preferably in the range of ½-2″. A mat or mattress (not shown) may be provided so the user can be comfortable while reclining in the booth 10.

The transparent cover or lid 14 is capable of transmitting most frequencies of sunlight so a session inside the booth 10 is not substantially different than a session of comparable length in direct sunlight. The lid 14 may be made of any suitable transparent or translucent material such as glass, plexiglass and a wide variety of organic polymers such as acrylic. The lid 14 may be of one piece formed on a mold or may be of multiple pieces glued together. The lid 14 can include a generally cylindrical top 40 and a pair of upright ends 42. Any suitable means may be provided to ventilate the booth 10 such an adjustable shutter 44 closing an opening 46 on each upright end 42 of the lid 14. The shutters are pivotally mounted on an axis 48 by a suitable fastener. By manipulating the shutters 44, which may be done from the inside of the booth 10, it is easy to control the amount of air passing through the booth 10 and thereby substantially affect the temperature inside the booth 10.

As a practical matter, the cover 14 is made of materials which are not overly strong. Economics dictate that the cover 14 be relatively thin and of an organic polymer. Such materials tend to warp or distort in response to forces acting on the cover 14. These forces may be generated by wind, thermal expansion and/or contraction of the cover material, the weight of the cover 14 and the like.

In some embodiments, the periphery of the cover 14 may be deformed during manufacture to provide a stronger rim. This may be accomplished by doubling over the material of the periphery, providing ridges in the periphery or the like. In a preferred embodiment, a subframe 50 is provided which is affixed to the rim or edge 52 of the cover 14. The subframe 50 is made of long parallel structural sections 54 welded or otherwise secured to short sections 56 in a box shape of the same length and width as the frame 12. The subframe 50 may be of the same metal as the frame 14 and aluminum is a preferred material.

Although the rim 52 may connect to the subframe 50 in a variety of techniques, one preferred arrangement is shown in FIGS. 3 and 4. The structural sections 54, 56 are illustrated as of box shape having a flat bottom surface 58, 60 overlapping the ends of the planar panel 32. One or more internal gussets 62, 64, 66, 68 divide the interior of the sections 54, 56 into several compartments, at least one compartment 70, 72 opening through a slot 74, 76 to receive the edge or rim 52. The edge 52 is secured in the compartments 70, 72 by an adhesive 78 of any suitable type such as a silicone caulk adhesive. A foam rope or backer rod 80 may be inserted into the compartments 70, 72 to reduce the quantity of silicone adhesive.

In a closed position of the lid 14, the subframe 50 sits on the edge of the planar panel 32 and is supported by the planar upper surface 20 of the frame 12. The frame 12 has no upstanding lip so the user doesn't have to contend with one when entering or sitting on the edge of the booth 10.

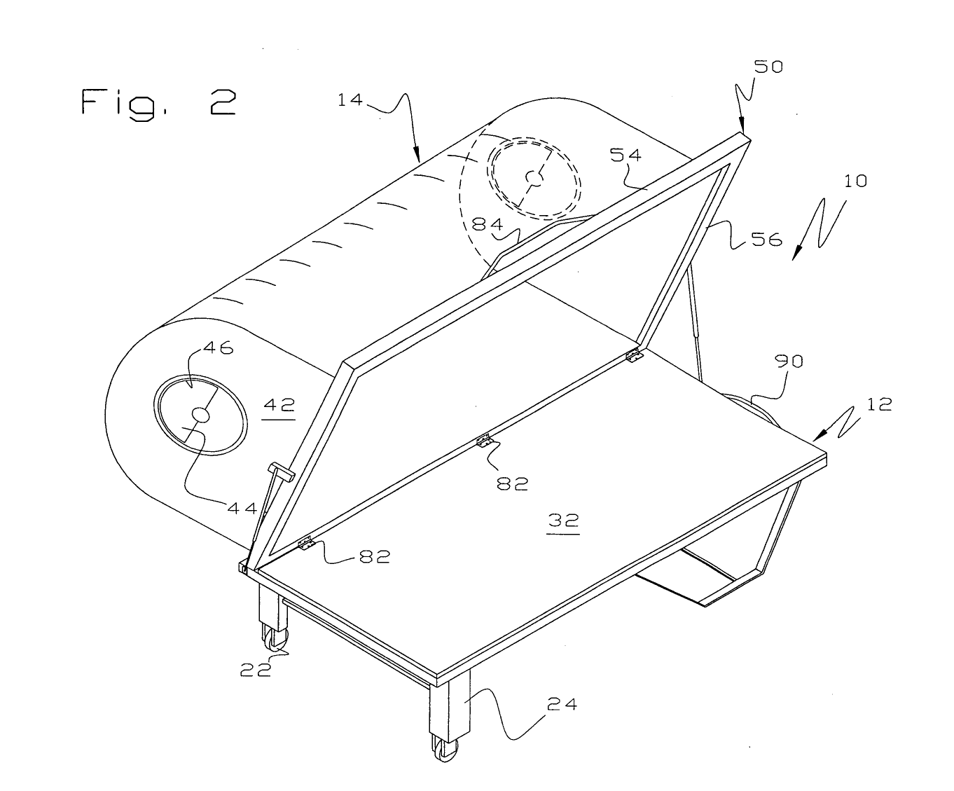

As shown in FIGS. 1-2, the subframe 50 may be connected to the frame 12 in any suitable manner, as by two or more hinges 82 so a user may grasp a handle 84 on the outside of the cover 14 or a handle 86 on the inside of the cover 14 and raise the cover from a closed position to an open position as shown by a comparison of photos 1 and 2. To partly overcome the weight of the cover 14, an air or gas cylinder 88 or other suitable counterweight can be provided on one or both ends of the booth 10. The gas cylinders 88 are of any suitable type such as are available from Grainger Corp. of Tampa, Fla. In this manner, anyone can raise and lower the cover 14 without undue exertion. A handle 90 on an end away from the wheels 24 allows a user to raise one end of the frame 12 and push or pull the booth 10 as it rolls on the underlying surface.

As disclosed hereinafter, an interesting advantage of the tanning booth 10 lies in the removable nature of the lid 14. To this end, the hinges 82 can be of a quick disconnect type, such as is available from McMaster-Carr Supply Company of Elmhurst, Ill. and the gas cylinders 88 can include quick disconnect fittings 92 so the gas cylinders 88 can be readily detached from the frames 12, 50. This allows the lid 14 to be easily removed.

Use of the solar powered tanning booth 10 should now be apparent. A user may elevate one end of the booth 10 using the handle 90 and push or pull the booth 10 to a desired location. The user may then grasp the handle 84 and raise the cover 14 to an open position so the user can enter the booth 10. The cover 14 is easily closed from inside the booth 10, partly due to the assistance of the gas cylinders 88. The shutters 44 may be adjusted to control the amount of air passing through the booth 10 to achieve a comfortable temperature. A user can then enjoy sunlight passing through the transparent lid 14 in moderate temperatures in quite cold weather because the booth 10 acts much like a greenhouse.

The lid 14 is readily removable which provides two additional techniques for using the components of the booth 10. In warm weather, e.g. atmospheric temperatures above about 75° F., it becomes unduly warm under the lid 14. By removing the lid 14, a person can use the frame 12 on which to recline and enjoy the warm weather and achieve tanning by direct sunlight. In cold weather, e.g. approaching freezing atmospheric temperature, or when wind conditions might suggest protecting fragile plants, the lid 14 can be removed and placed over temperature sensitive plants so the lid 14 acts much like a small greenhouse. The lid 14 can also be used, in warm, cool or cold weather, as a nursery to start or nurture young plants grown from cuttings or seedlings grown from germinated seeds. Thus, the tanning booth 10 can be used in its normal fashion during cool or cold weather and its components can be separated and then used in either cold or warm weather. The reverse is also true, i.e. the frame 12 and lid 14 can be used separately and then joined for tanning use in cold weather.

Although this invention has been disclosed and described in its preferred forms with a certain degree of particularity, it is understood that the present disclosure of the preferred forms is only by way of example and that numerous changes in the details of operation and in the combination and arrangement of parts may be resorted to without departing from the spirit and scope of the invention as hereinafter claimed.

We claim:

1. A solar powered tanning booth comprising
a frame having a peripheral structural member providing a flat upper surface free of an upstanding lip, supports elevating the frame off an underlying ground surface and a panel spanning the structural member providing a horizontal support for a person; and
a lid having a transparent dome allowing sunlight to pass therethrough, a peripheral structural member providing a flat lower surface, a hinge joining the lid and the frame and pivoting the lid from an open position providing access to an interior of the booth and a closed position where the flat lower surface of the lid structural member overlies and is supported by the flat upper surface of the frame structural member;
the lid peripheral structural member providing an upwardly opening slot receiving a lower edge of the lid and an adhesive securing the lid in the slot.

2. The solar powered tanning booth of claim 1 further comprising wheels on at least some of the supports.

3. The solar powered tanning booth of claim 1 wherein the panel is a normally planar panel extending to edges of the frame structural member and the lid structural member overlies and rests on the panel when the lid is in the closed position.

4. The solar powered tanning booth of claim 3 further comprising at least one brace extending across the frame structural member and underlying the planar panel, the panel normally being out of contact with brace and being deformed into load supporting engagement with the brace upon a person lying on the panel.

5. A solar powered tanning booth comprising
a frame having a peripheral structural member having an upper surface and including a long side and a short side, supports elevating the frame off an underlying ground surface, a normally planar panel spanning the structural member, having a bottom flush with the frame upper surface and a top providing a horizontal support for a person and a brace connecting the long sides of the structural member and lying below and out of engagement with the bottom of the panel, loading of the panel acting to deform the panel in load supported engagement with the brace, the panel being upwardly concave upon loading; and
a lid having a transparent dome allowing sunlight to pass through the lid and a hinge joining the lid and the frame and pivoting the lid from a first closed position where the lid is supported by the frame structural member to a second open position providing access to an interior of the booth.

6. The solar powered tanning booth of claim 5 further comprising wheels on at least some of the supports.

7. The solar powered tanning booth of claim 5 wherein the panel is a normally planar panel extending to edges of the frame structural member and the lid structural member overlies and rests on the panel when the lid is in the closed position.

8. A method of using a tanning bed including a body support and a transparent lid, comprising
delivering solar energy to a person on the support under the lid in a time of cold atmospheric temperature, and
removing the lid and then delivering solar energy directly to a person on the support in a time of warm atmospheric temperature.

9. The method of claim 8 further comprising, after the removing step, placing the lid over a temperature sensitive plant in a time of approaching freezing weather.

10. A method of using a tanning bed including a body support and a transparent lid, comprising
delivering solar energy to a body on the support under the lid in a time of cold atmospheric temperature,
removing the lid and then placing the lid over a temperature sensitive plant.

11. The method of claim 11 wherein the placing step occurs in a time of cold atmospheric temperature.

12. The method of claim 11 wherein the temperature sensitive plant is a seedling.

13. The method of claim 11 wherein the temperature sensitive plant is a cutting.

14. The method of claim 11 wherein the temperature sensitive plant comprises a young plant germinated from a seed.
